(19) United States
(12) Patent Application Publication
(10) Pub. No.: US 2026/0345030 A1
(43) Pub. Date: Nov. 5, 2026

(54) CLINICAL MONITORING TECHNIQUE USING PASSIVE INFRARED SENSORS

(71) Applicant: HYPROS GMBH, Stralsund (DE)

(72) Inventors: Tobias GEBHARDT, ROSTOCK (DE); Marcel WALZ, Stralsund (DE)

(21) Appl. No.: 19/113,270

(22) PCT Filed: Sep. 30, 2022
(86) PCT No.: PCT/EP2022/077380

§ 371 Date: Mar. 19, 2025

(51) Int. Cl. A61B 5/11; A61B 5/00; A61B 5/01

(52) U.S. Cl. CPC A61B 5/1113; A61B 5/0059; A61B 5/01; A61B 5/1126; A61B 2562/043

(57) ABSTRACT

We disclose a clinical monitoring device, comprising: a plurality of passive infrared, PIR, sensors for monitoring a patient zone; and an arrangement configured to define a plurality of detection areas for the PIR sensors, wherein at least one of the detection areas is associated with two or more of the PIR sensors, wherein the detection areas associated with a respective one of the PIR sensors form a group of detection areas covering an elongate spatial area extending longitudinally along a spatial axis, wherein the spatial axis differs between two or more of the groups of detection areas. Also disclosed herein are clinical monitoring system comprising the device, a clinical monitoring kit comprising the device, a clinical monitoring bundle comprising the device, a related method using the device, a corresponding computer program and a carrier carrying the computer program.

### TECHNICAL FIELD

The present disclosure generally relates to a clinical monitoring device, a clinical monitoring method, a clinical monitoring kit, a clinical monitoring bundle, a clinical monitoring system and a computer program.

### BACKGROUND

In hospitals, care beds, also referred to as patient beds herein, are usually located in patient zones in designated patient rooms. A patient may enter a certain care bed at a first point in time. A caregiver such as a nurse or a doctor may enter the patient zone to visit the patient lying in the care bed at a later second point in time. The patient in the care bed may receive additional visitors later on, who may or may not enter the patient zone. The patient may leave the care bed at a later third point in time. The care bed may be remade (e.g., for a next patient) by clinical staff at a fourth point in time.

It may be useful to monitor the patient zone to obtain information about some or all of the above activities that take place in the patient zone. For example, such monitoring of the patient zone may be used for determining whether a care bed is occupied, whether clinical personnel must disinfect their hands (e.g., after an interaction with the patient in the care bed), whether a care bed has been remade and the like.

Known monitoring techniques used in different fields such as security surveillance include use of video cameras and object recognition algorithms to detect persons in certain areas. Such solutions suffer from the drawback of expensive hardware, high power consumption, high required data transmission rates and a lack of protection of personal data.

### SUMMARY

There is a need for a clinical monitoring technique that solves one or more of the aforementioned or other problems.

According to a first aspect, a clinical monitoring device is provided. The device comprises a plurality of passive infrared, PIR, sensors for monitoring a patient zone, and an arrangement configured to define a plurality of detection areas for the PIR sensors. At least one of the detection areas is associated with two or more of the PIR sensors, wherein (e.g., all) the detection areas associated with a respective one of the PIR sensors form a group of detection areas covering an elongate spatial area extending longitudinally along a spatial axis, wherein the spatial axis differs between two or more of the groups of detection areas.

The patient zone may be a spatial zone in which a patient is (e.g., regularly or expected to be) present. The patient zone may be a spatial zone in which (e.g., a portion of) a care bed or patient bed is located. The patient zone may be larger than the bed. The patient zone may cover the bed and a surrounding or bounding region of the bed.

Each PIR sensor may be a pyroelectric infrared sensor. A PIR sensor may also referred to as a passive infrared detector, PID. Each PIR sensor may be configured to not radiate energy for detection purposes but to detect infrared radiation (e.g., radiant heat) emitted by or reflected from objects or living beings. The clinical monitoring device may comprise a housing and the plurality of PIR sensors may be arranged on or within the housing.

Each PIR sensor may be configured to detect a change of temperature within a (e.g., three-dimensional) field of view, FoV. The FoVs may be sensor-specific and defined, at least in part, by the arrangement. A detection area of a PIR sensor may be considered to be the two-dimensional intersection plane through the FoV of the PIR sensor, which intersection plane is normal or orthogonal to a central or main viewing axis of the PIR sensor. The central viewing axis may correspond to an axis extending longitudinally in a center of the FoV. In case the clinical monitoring device is mounted on the ceiling and the PIR sensors are directed toward the floor, the detection areas may be areas on the floor. A detection area may be, may have an outer contour, or may have a shape that is triangular, rectangular, square, hexagonal, circular or rounded. A detection area may abut, be adjacent to, border or adjoin another detection area (e.g., associated with the same or a different PIR sensor). A detection area may be distant from still another detection area (e.g., associated with the same or a different PIR sensor). In case a detection area is observed by (e.g., monitored by or visible to) only one PIR sensor, it is associated only with this one PIR sensor. In case a detection area is observed by exactly two PIR sensors, it is associated with these two PIR sensors.

The group of detection areas covers an elongate spatial area, which means that all detection areas of this group fall into the elongate spatial area. The elongate spatial area may consist of the detection areas of the group or may comprise at least all the detection areas of the group. The elongate spatial area may be referred to as an outer contour of all detection areas of the group. The elongate spatial area may be a (e.g., rectangular) bounding box comprising all of the detection areas of the group (e.g., and as few other spatial areas or detection areas as possible). The elongate spatial area may have a main axis (e.g., a central axis or an axis defined by the longest extension direction of the elongate spatial area) that corresponds to the spatial axis along which the elongate spatial area extends longitudinally.

The groups of detection areas of the two or more of the PIR sensors that are associated with the at least one of the detection areas may cover different elongate spatial areas. The spatial axes of the groups of detection areas of the two or more of the PIR sensors that are associated with the at least one of the detection areas may differ from one another. The spatial axes of the groups of detection areas of the two or more of the PIR sensors that are associated with the at least one of the detection areas may be (e.g., essentially) orthogonal to one another. In one variant, each group of detection areas covers a different elongate spatial area. In this variant, one may say that each of the PIR sensors observes a different elongate spatial area.

The spatial axes of a first set of the groups may be (e.g., essentially) parallel to one another. The spatial axes of a second set of the groups may be (e.g., essentially) parallel to one another and (e.g., essentially) non-parallel to the spatial axes of the first set. The spatial axes of the first set may be (e.g., essentially) orthogonal to the spatial axes of the second set. The detection areas may be arranged in a (e.g., periodic, uniform or homogeneously distributed) grid pattern. All detection areas may be arranged in or form a common grid pattern having columns and rows. At least a part of the patient zone may be covered with or by the detection areas. For example, the patient zone may be fully covered with or by the detection areas.

Each of the detection areas may be associated with exactly one or exactly two of the PIR sensors. Each of the detection areas may be associated with a different subset (e.g., pair or triplet) of the PIR sensors. The association between the detection areas and the PIR sensors may be predefined (e.g., known and/or stored on a storage medium). The number of detection areas may be larger than the number of PIR sensors. The number of the at least one detection area associated with two or more of the PIR sensors may be larger than the number of PIR sensors. The number d of the at least one detection area associated with two or more of the PIR sensors may be d=a×b, wherein the number s of PIR sensors may be s=a+b. A spatial arrangement of the PIR sensors may differ from a spatial arrangement of the groups of detection areas. For example, the PIR sensors may be arranged in columns and rows and the detection areas may be arranged in columns and rows, wherein a number of columns and/or a number of rows differs between the PIR sensors and the detection areas.

A group of detection areas may consist of detection areas that cover a continuous or coherent spatial portion. In this case, the detection areas of that group may be adjacent to one another, border one another and/or be joined together. Alternatively, a group of detection areas may consist of detection areas that cover an interrupted spatial portion of a plurality of distinct and/or disjoint spatial portions. In this case, a first subgroup of the detection areas of that group may be spatially offset from a second subgroup of the detection areas of that group.

The detection areas may cover the complete patient zone. Alternatively, an overall spatial detection range of the PIR sensors may comprise blind spots or blind regions that are not covered by the detection areas. Put differently, certain parts of the patient zone may be (e.g., intentionally) blocked out such that they are not observable with the PIR sensors of the clinical monitoring device. For example, a portion of the patient zone in which the patient bed, a window, a heater and/or electrical medical equipment is located may be blocked out such that the detection regions do not cover this blocked out portion. Alternatively, or in addition, a region bordering or bounding the patient bed may be blocked out.

The arrangement may comprise one or more (e.g., optical) apertures. The arrangement may comprise, for each of the PIR sensors, (e.g., at least or exactly) one aperture. Each of the apertures may be associated with (e.g., a different) one of the PIR sensors and define at least an outer contour of (i) (e.g., one or more of) the detection areas associated with the one PIR sensor or (ii) the group of detection areas associated with the one PIR sensor. Each aperture may have an overall (e.g., elongate and/or rectangular) slot-shape. Each aperture may be arranged in a predefined position and orientation relative to the associated PIR sensor. All apertures (e.g., of a subset of the apertures) may have the same shape. One or more (e.g., all) of the apertures may have a spatial offset from a central detection axis (e.g., a central or main viewing direction) of the PIR sensor associated with the respective aperture. All apertures (e.g., of a first set of the apertures) may differ from one another in the spatial offsets. All apertures may have the same shape. A first subset of the apertures may differ from a second subset of the apertures (e.g., only) in an orientation of the apertures and/or in the spatial offsets. At least one of the PIR sensors may be associated with two or more apertures. A spatial offset (e.g., distance) between adjacent apertures may be larger than a spatial offset (e.g., distance) between the (e.g., central viewing axes of the) associated adjacent PIR sensors.

One or more of the apertures may be adjustable in size, shape (e.g., contour) and/or position (e.g., relative to the associated PIR sensor). The arrangement may comprise at least one actuator (e.g., a motor) configured to adjust the one or more of the apertures in size, shape and/or position relative to the associated PIR sensor.

The arrangement may comprise a sheet in which one or more of the apertures is formed as a (e.g., rectangular) through-hole. Portions between the through-holes may define the blind spots or blind regions (e.g., by blocking infrared light originating from these blind spots or blind regions within the patient zone form reaching one or more of the PIR sensors). Each through-hole may have the same size, shape (e.g., contour or outline), position and/or orientation as (i) the elongate spatial area associated with the respective PIR sensor, (ii) the group of detection regions associated with the respective PIR sensor and/or (iii) at least one of the detection regions associated with the respective PIR sensor.

The device may comprise a plurality of sheets, each comprising one or more through-holes, wherein the through-holes of the sheets overlap at least partially, the overlapping portion defining the size, shape, position and/or orientation of (i) the elongate spatial area associated with the respective PIR sensor, (ii) the group of detection regions associated with the respective PIR sensor and/or (iii) at least one of the detection regions associated with the respective PIR sensor.

The sheet(s) may (e.g., each) be configured as a replaceable component of the clinical monitoring device. The sheet(s) may be configured to be replaceably mounted to the device 2 in a predefined pose relative to PIR sensors 46. The sheet(s) may be opaque for infrared light (e.g., transmit less than 10%, less than 5% or less than 1% of infrared light (e.g., at wavelengths between 8 μm and 10.5 μm)).

The arrangement may further comprise a light-blocking assembly configured to block light falling in through one of the apertures from reaching one or more of the PIR sensors that are not associated with the one of the apertures. The light-blocking assembly may comprise one or more walls configured to extend between adjacent PIR sensors. The one or more walls may be opaque for infrared light (e.g., block infrared light). The light-blocking assembly may be fixedly arranged relative to the at least one of (i) one or more of the apertures, or (ii) one or more of the PIR sensors. The light-blocking assembly may be attached to (e.g., one of) the sheet(s) or formed together with the (e.g., one) sheet as a single component (e.g., by injection-molding).

The clinical monitoring device may further comprise a protective cover configured to protect the PIR sensors from an outer environment. The protective cover may be arranged within or across a field of view of one or more (e.g., all) of the PIR sensors. The protective cover may be arranged to cover one or more (e.g., all) of the apertures. The protective cover may be arranged on (e.g., adhesively attached to) the sheet. The protective cover may be transparent for infrared light (e.g., configured to transmit more than 60% or more than 70% of infrared light at wavelengths between 8 μm and 10.5 μm). The protective cover may be arranged such that the PIR sensors (e.g., and any other sensors comprised in the clinical monitoring device) are not visible (e.g., to the human eye) from an outside of the clinical monitoring device. The transparent cover may be made of (e.g., high density, HD) polyethylene, PE. The transparent cover may be a HDPE foil.

The arrangement may comprise one or more (e.g., Fresnel) lenses. The arrangement may comprise two or more lenses, each being associated with a different one of the PIR sensors. Each lens may be arranged in a predefined position and orientation relative to the associated PIR sensor. Each lens may comprise a receptacle in which the associated PIR sensor is located. The arrangement may comprise a lens carrier, wherein the two or more lenses are fixedly arranged relative to one another on the lens carrier. The two or more lenses may be attached to the lens carrier or manufactured together with the lens carrier as a single component. At least one of the one or more lenses may be associated with two or more of the PIR sensors. In other words, a common lens may be provided for the two or more of the PIR sensors. The arrangement may comprise exactly one lens, wherein the lens is associated with each of the PIR sensors. The lens(es) (e.g., together with the one or more apertures) may define a size, shape, position and/or orientation of (i) the elongate spatial area associated with the respective PIR sensor, (ii) the group of detection regions associated with the respective PIR sensor and/or (iii) at least one of the detection regions associated with the respective PIR sensor.

The clinical monitoring device may further comprise a mounting arrangement configured to tiltably mount the (e.g., housing of the) device (e.g., rotatable around a mounting axis) to a ceiling or wall of a hospital room such that the detection areas can be shifted relative to the hospital room by tilting the device. Shifting the detection areas may result in a (e.g., trapezoidal) deformation of the detection areas. the housing of the clinical monitoring device may be configured to be detachable from the mounting arrangement (e.g., for maintenance or battery replacement).

The clinical monitoring device may comprise a flexible substrate (e.g., a flexible circuit board) carrying the PIR sensors. The arrangement may be configured to define (e.g., a spatial alignment of) a central detection axis of each of the PIR sensors such that the central detection axes of at least two (e.g., neighboring or adjacent ones) of the PIR sensors differ from one another (e.g., are non-parallel or oblique to one another). The arrangement may comprise a (e.g., replaceable) shape-defining member (e.g., a wedge, a half-cylinder or a cylinder portion) configured to deform the flexible substrate into a predefined shape. The shape-defining member may be configured to deform the flexible substrate with a predefined bending radius such that an angular offset is defined between the central detection axes of the (e.g., adjacent) PIR sensors.

The clinical monitoring device may further comprise one or more additional sensors. In one variant, the clinical monitoring device may comprise an additional PIR sensor having FoV covering a spatial portion adjacent to at least some of the outermost detection areas of the plurality of PIR sensors. The FoV of the additional PIR sensor may extend beyond the patient zone, for example cover a region around one or more sides of the patient zone. The additional PIR sensor may be configured as a wake-up sensor to activate one or more components of the clinical monitoring device from a standby state to an active state upon detection of a temperature change within the FoV which exceeds a predefined threshold (e.g., a minimum detection threshold of the additional PIR sensor).

The clinical monitoring device may further comprise a thermal sensor arrangement configured to provide spatially resolved temperature measurements of a spatial sector comprising at least one of the detection areas. The spatial sector may have a predefined spatial relationship to one or more of the detection areas. The spatial sector may comprise all detection areas. The spatial sector may cover the complete patient zone.

The clinical monitoring device may further comprise a distance sensor arrangement configured to provide spatially resolved distance measurements of a spatial field comprising at least one of the detection areas. The spatial field may have a predefined spatial relationship to one or more of the detection areas. The spatial field may comprise all detection areas. The spatial field may cover the complete patient zone. The spatial field may correspond to the spatial sector.

The clinical monitoring device may further comprise a wired or wireless interface configured to output data based on signals of the plurality of PIR sensors (e.g., and at least one of the temperature measurements and the distance measurements).

According to a second aspect. A clinical monitoring method is provided. The method uses a clinical monitoring device comprising (i) a plurality of passive infrared, PIR, sensors for monitoring a patient zone, and (ii) an arrangement configured to define a plurality of detection areas for the PIR sensors, wherein at least one of the detection areas is associated with two or more of the PIR sensors, wherein the detection areas associated with a respective one of the PIR sensors form a group of detection areas covering an elongate spatial area extending longitudinally along a spatial axis, wherein the spatial axis differs between two or more of the groups of detection areas. The method may use the clinical monitoring device of the first aspect. The method comprises obtaining, from at least one of the PIR sensors, a detection signal indicative of a temperature change exceeding a predefined (e.g., detection) threshold, and identifying, based on the obtained detection signal(s), a spatial region in which the temperature change exceeding the predefined threshold occurred.

The method may be performed by at least one processor (e.g., part of the clinical monitoring device or separate therefrom). The detection signal(s) may be directly obtained by the at least one processor from the at least one of the PIR sensors, or from the clinical monitoring device via the wired or wireless interface.

The detection signals may be obtained from the two or more of the PIR sensors associated with the at least one of the detection areas and may be indicative of a temperature change within the at least one of the detection areas exceeding the predefined (e.g., detection) threshold. In this case, the at least one of the detection area may be identified as (e.g., at least a part of) the spatial region. A temperature change within a FoV of a PIR sensor (e.g., between the detection area and the associated PIR sensor) may be considered to be a temperature change within that detection area.

The temperature change indicated by the obtained detection signals may be observed by the two or more of the PIR sensors within a (e.g., first) predefined time window. For example, the at least one of the detection areas may be identified as (e.g., at least a part of) the spatial region only if the detection signals indicate that the PIR sensors associated with the at least one of the detection areas detected the temperature change within the predefined (e.g., first) time window (e.g., no more than a (e.g., first) predefined temporal difference apart from one another).

A detection, by multiple sensors and within the (e.g., first) time period (e.g., and for a particular detection area), of a temperature change exceeding the (e.g., same or sensor-specific) predefined threshold may be referred to as a simultaneous detection. The first predefined time window may be used for determining or detecting simultaneous detections within a particular detection area. The spatial region may comprise or consist of (e.g., all) detection areas in which simultaneous detections were detected within a predefined (e.g., second) time window. The second predefined time window may be used for selecting all the detection areas in which the simultaneous detections were observed, which simultaneous detections fall into the same second time window. The spatial portion may comprise or consist of the so-selected detection areas. In a first step, the detection signals may be used to determine one or more simultaneous detections using the first time window. In a second step, the determined simultaneous detections may be used to identify the spatial region by selecting (e.g., grouping) all detection areas associated with simultaneous detections as part of the spatial region. The first predefined time window may be (e.g., two to fifteen times, for example three to ten times, or four to ten times) smaller (e.g., shorter) than the second predefined time window.

The spatial region may be identified for different points in time (e.g., by moving the first time window and the second time window according to an elapsed time across detection signals of the PIR sensors). The detection signals may be continually analyzed using the first and the second time window, for example by detecting peaks falling within the first time window as simultaneous detections. Detection areas associated with these simultaneous detections may then be dynamically grouped into the spatial region depending on whether or not the simultaneous detections fall into the second time window. The method may comprise determining a movement of a heat-dissipating object or living being (e.g., a human or an animal) based on a change of the identified spatial region over time. The movement may be determined by tracking a location of a center of the identified spatial region over time.

The method may further comprise obtaining spatially resolved temperature measurements of a (e.g., the) spatial sector comprising at least one of the detection areas, and categorizing (e.g., as a patient (e.g., a patient having left the patient bed), as a visitor (e.g., a visitor located next to the patient bed) or as a heat-dissipating object) the identified spatial region or parts of the identified spatial region based on the spatially resolved temperature measurements. The categorizing may comprise spatially mapping or matching the spatially resolved temperature measurements to the detection signal(s) obtained from the at least one PIR sensor. The clinical monitoring device may comprise a thermal sensor arrangement configured to provide the spatially resolved temperature measurements, as described above for the first aspect. Alternatively, the spatially resolved temperature measurements may be obtained from a different entity (i.e., not from the clinical monitoring device).

The method may further comprise obtaining spatially resolved distance measurements of a (e.g., the) spatial field comprising at least one of the detection areas, and categorizing (e.g., as a patient lying in the patient bed, as a visitor having entered the patient zone, or as an object present within the patient zone and exhibiting a temperature change) the identified spatial region or parts of the identified spatial region based on the spatially resolved distance measurements. The categorizing may comprise spatially mapping or matching the spatially resolved distance measurements to the detection signal(s) obtained from the at least one PIR sensor. The clinical monitoring device may comprise a distance sensor arrangement configured to provide the spatially resolved distance measurements of the spatial field, as described above for the first aspect. Alternatively, the spatially resolved distance measurements may be obtained from a different entity (i.e., not from the clinical monitoring device).

The method may comprise determining (e.g., the presence of) an event based on the identified spatial region and, optionally, at least one of (i) the spatially resolved temperature measurements and (ii) the spatially resolved distance measurements. The event may be indicative of at least one activity selected from: a person entering the patient zone, a person being present next to a patient bed located in the patient zone, a patient lying in the patient bed, a patient entering the patient bed, a patient leaving the patient bed, a person leaving the patient zone, and a person being in close proximity to the patient (e.g., the patient lying in the patient bed), medical equipment within the patient zone heating up, a heater or radiator within the patient zone heating up, or a window shutter being opened. The event may be indicative of other activities or of a plurality of activities. The event may be determined by spatially mapping or matching the spatially resolved temperature and/or distance measurements to the detection signal(s) obtained from the at least one PIR sensor or to the detection region(s) for which the detection signal(s) indicate detection of a temperature change that exceeds the predefined threshold. One or more trained machine learning models may perform at least one of the categorization and the determination of the event.

The method may comprise, based on the determined event(s), determining a recommended action (e.g., that the person having entered the patient zone needs to perform a hygiene activity (e.g., disinfect his/her hands), that the patient bed needs to be remade, that clinical personnel should visit the patient (e.g., if he/she has fallen out of the patient bed), or the like). The determined event and/or the recommended action may be output, for example on a display for a user.

The method may further comprise detecting, with an (e.g., the) additional passive infrared sensor, PIR, having a field of view covering a spatial portion adjacent to at least some of the outermost detection areas of the plurality of PIR sensors, a temperature change in the field of view of the additional PIR sensor exceeding a predefined threshold. The method may comprise, responsive to the detecting, activating one or more components of the clinical monitoring device from a standby state to an active state. The clinical monitoring device may comprise the additional passive infrared sensor as described above for the first aspect. Alternatively, the additional passive infrared sensor may be part of a different entity (i.e., not of the clinical monitoring device).

According to a third aspect, a clinical monitoring system is provided. The clinical monitoring system comprises the clinical monitoring device according to the first aspect and a computing system configured to perform the method according to the second aspect. The computing system may be communicatively coupled to the clinical monitoring device (e.g., via the wired or wireless interface thereof). The computing system may be part of a hospital management system. The computing system may be implemented as a (e.g., distributed) cloud-computing system.

The computing system may comprise at least one processor configured to perform the method according to the second aspect. Alternatively, or in addition, the clinical monitoring device according to the first aspect may comprise at least one processor configured to perform the method according to the second aspect.

According to a fourth aspect, a clinical monitoring kit is provided. The clinical monitoring kit comprises the clinical monitoring device of the first aspect and a plurality of the sheets, differing from one another in at least one of (i) a number of apertures formed in the respective sheet, (ii) a shape (e.g., contour or outline) of one or more apertures formed in the respective sheet, (iii) a size of one or more apertures formed in the respective sheet, (iv) a position and/or orientation of one or more apertures formed in the respective sheet. The sheets of the kit may alternatively or additionally differ from one another in the (e.g., degree or type of) adjustability of one or more of the apertures formed in the respective sheet. Each of the sheets may be specific for a particular type of patient bed or use scenario. Each of the sheets may define different detection areas or different groups of detection areas (e.g., differing from sheet to sheet in shape, size, position and/or orientation).

According to a fifth aspect, a clinical monitoring bundle is provided. The clinical monitoring bundle comprises the clinical monitoring device of the first aspect and a plurality of the shape-defining members, differing from one another in the predefined shapes into which they deform the flexible substrate. Each of the shape-defining members may be specific for a particular type of patient bed, patient zone or use scenario.

According to a sixth aspect, a computer program is provided. The computer program comprises instructions which, when executed by at least one processor, cause the at least one processor (e.g., of the computing system of the third aspect or of the clinical monitoring device of the first aspect) to perform the method according to the second aspect.

According to a seventh aspect, a carrier is provided. The carrier carries or stores the computer program of the sixth aspect. The carrier may be a memory, a non-transitory computer readable storage medium or a data stream. The carrier may be part of the clinical monitoring system of the third aspect. Alternatively, or in addition, the clinical monitoring device of the first aspect may comprise such a carrier.

### BRIEF DESCRIPTION OF THE DRAWINGS

Further details, advantages and aspects of the present disclosure will become apparent from the following embodiments taken in conjunction with the drawings, wherein:

FIG. 1 shows a clinical monitoring device arranged within a hospital room;

FIG. 2 shows a clinical monitoring system comprising the clinical monitoring device;

FIG. 3 illustrates exemplary detection areas in accordance with the present disclosure;

FIG. 4a-4c illustrate exemplary detection areas in accordance with the present disclosure;

FIG. 5 shows an explosion drawing of the clinical monitoring device;

FIG. 6 shows a schematic sideview of a PIR sensor of the clinical monitoring device;

FIG. 7a-7b show different sheets for the clinical monitoring device;

FIG. 8 shows an arrangement of the clinical monitoring device;

FIG. 9 shows a flexible substrate of the clinical monitoring device;

FIG. 10 shows a perspective view of the clinical monitoring device;

FIG. 11 shows a method in accordance with the present disclosure;

FIG. 12a-12b visualize sensor data in accordance with the present disclosure; and

FIG. 13a-13f illustrate determination of a movement based on PIR sensor data in accordance with the present disclosure.

### DETAILED DESCRIPTION

In the following description, exemplary embodiments will be explained with reference to the drawings. The same reference numerals will be used to denote the same or similar structural or functional features.

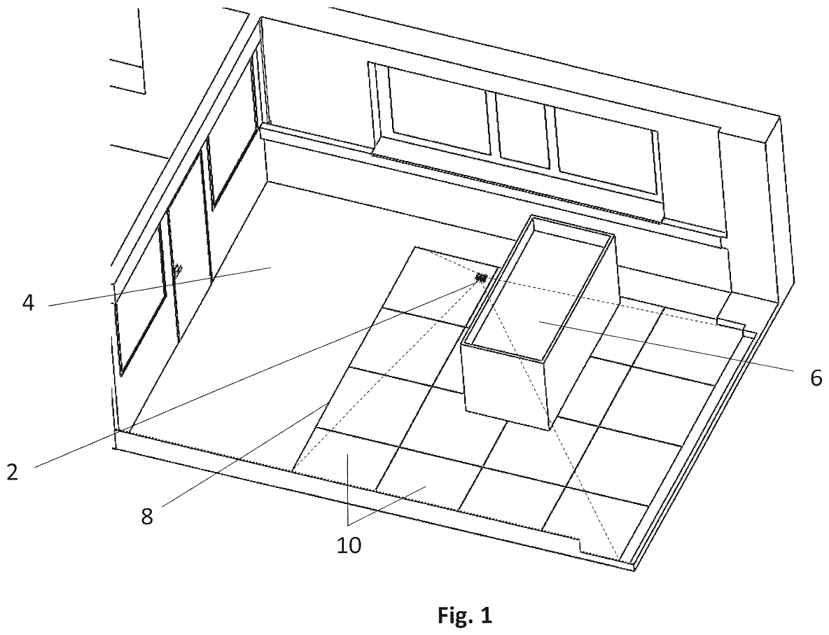

FIG. 1 illustrates a clinical monitoring device 2 arranged within a room 4 of a hospital. A care bed or patient bed 6 is also arranged within the room 4. The clinical monitoring device to comprises a plurality of passive infrared, PIR, sensors, for example pyroelectric infrared sensors, for monitoring a patient zone 8 in which the patient bed 6 is located. A plurality of detection areas 10 within the patient zone 8 are observed by the PIR sensors. In the illustrated example, one may say that each detection area 10 corresponds to a two-dimensional area on the floor of the room 4. The patient bed 6 in the illustrated example covers parts of six detection areas 10.

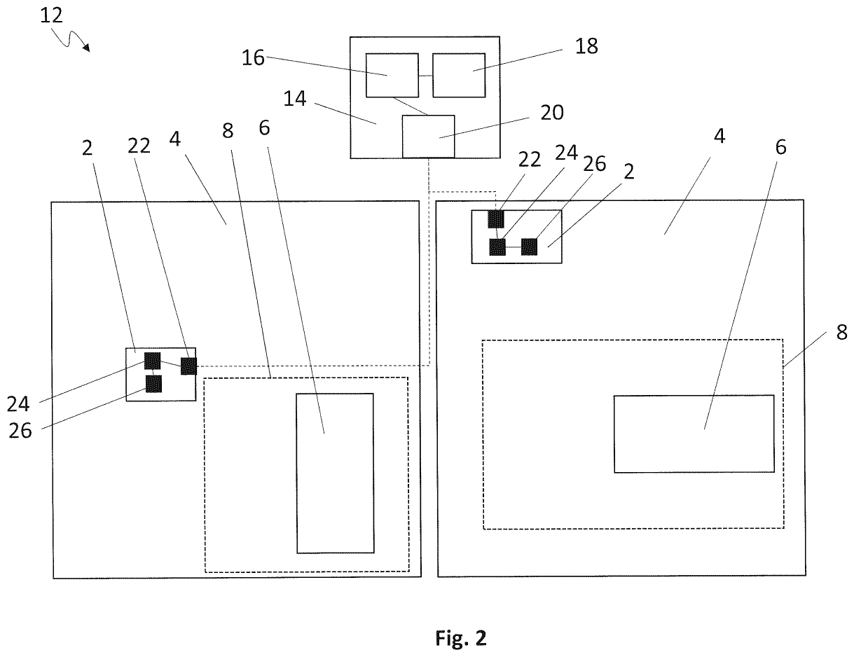

As shown in FIG. 2, a clinical monitoring system 12 may be provided. The clinical monitoring system 12 comprises the clinical monitoring device 2 and a computing system 14. The computing system 14 may be configured to perform the methods described herein. In the illustrated example, the clinical monitoring system 12 comprises a plurality of clinical monitoring devices 2, wherein at least two of the clinical monitoring devices 2 are each arranged in different hospital rooms 4. Each of the clinical monitoring devices 2 is configured to monitor a separate patient zone 8, in which a respective patient bed 6 is located. In the illustrated example, the computing system 14 comprises at least one processor 16 communicatively coupled to at least one memory 18 and at least one interface 20. The at least one processor 16 may be configured to perform the methods described herein, for example based on instructions stored on the at least one memory 18.

Each clinical monitoring device 2 may comprise a wired or wireless interface 22 communicatively coupled (e.g., via a Bluetooth connection, a radio connection, a cellular connection or a wireless local area network, WLAN, connection) to the computing system 14, for example via the at least one interface 20. Each clinical monitoring device 2 may comprise at least one processor 24. Furthermore, each clinical monitoring device 2 may comprise at least one memory 26. The at least one processor 24 may be configured to perform the methods described herein, for example based on instructions stored on the at least one memory 26. Each of the memories 18, 26 may be referred to as a carrier carrying a computer program comprising the instructions.

The clinical monitoring device 2 comprises an arrangement configured to define a plurality of detection areas 10 for the PIR sensors of the clinical monitoring device 2. At least one of the detection areas 10 is associated with two or more of the PIR sensors, which means that the at least one of the detection areas 10 is observed by the two or more of the PIR sensors (e.g., continuously and/or at the same time). Furthermore, the (e.g., all) detection areas 10 associated with one of the PIR sensors of the clinical monitoring device 2 form a group of detection areas. That is, each of the PIR sensors is (e.g., only) associated with detection areas 10 forming a (e.g., sensor-specific) group.

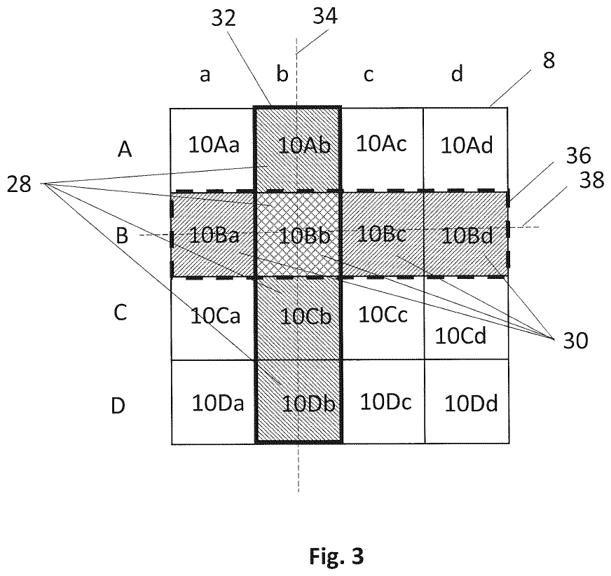

FIG. 3 illustrates exemplary groups of detection areas 10 that may be defined by the arrangement of the clinical monitoring device 2. In this example, the detection areas 10 are arranged in a periodic, square grid pattern of equally sized columns a-d and equally sized rows A-D. All detection areas 10Ab, 10Bb, 10Cb and 10Db of the column b form a group 28 of detection areas 10 associated with a first of the PIR sensors. All detection areas 10Ba, 10Bb, 10Bc and 10Bd of the row B form a group 30 of detection areas 10 associated with a second of the PIR sensors. As can be seen, the group 28 of detection areas 10Ab, 10Bb, 10Cb and 10Db covers an elongate spatial area 32 which is, in the illustrated example, a rectangular boundary surrounding the detection areas 10Ab, 10Bb, 10Cb and 10Db comprised in the group 28. The elongate spatial area 32 extends longitudinally along a spatial axis 34. The group 30 of detection areas 10Ba, 10Bb, 10Bc and 10Bd forms an elongate spatial area 36 which is, in the illustrated example, a rectangular bounding box enclosing the detection areas 10Ba, 10Bb, 10Bc and 10Bd that make up the group 30. The elongate spatial area 36 has a spatial axis 38 as its longest axis of symmetry. The axes 34, 38 are orthogonal to one another.

The detection area 10Bb is comprised in both group 28 and group 30. That is, the detection area 10Bb is associated with both the first and the second of the PIR sensors of the clinical monitoring device 2, which means the spatial monitoring ranges of these two PIR sensors overlap in the detection area 10Bb. In case the first PIR sensor associated with group 28 detects a change of temperature and the second PIR sensor associated with group 30 does not detect a change in temperature, it can be assumed that the change in temperature occurred in one of the detection areas 10Ab, 10Cb or 10Db. In case the second PIR sensor associated with group 30 detects a change of temperature and the first PIR sensor associated with group 28 does not detect a change in temperature, it can be assumed that the change in temperature was present in one of the detection areas 10Ba, 10Bc or 10Bd. If both the first and the second PIR sensors, which are each associated with the detection area 10Bb, detect a change in temperature at essentially the same time (e.g., within a first predetermined time window), it can be assumed that the temperature change occurred in the detection area 10Bb. A change in temperature “in a detection area” may be equivalent to a change in temperature within the field of view, FoV, of the PIR sensor associated with this detection area (e.g., within a spatial volume between this PIR sensor and the detection area 10).

Each detection area 10 within a column a, b, c or d may be associated with a different one of the PIR sensors, and each detection area 10 within a row A, B, C or D may be associated with a still different one of the PIR sensors. In this case, the clinical monitoring device 2 may comprise a separate PIR sensor for each column a-d and a separate PIR sensor for each row A-D of the grid of detection areas 10. In the example of FIG. 3, this means that the device 2 would comprise a total of 4+4=8 PIR sensors to monitor each of the 4×4=16 detection areas 10. In this case, each detection area 10 would be associated with one PIR sensor covering a row of detection areas 10 and one PIR sensor covering a column of detection areas 10, which means each detection area 10 would be monitored by exactly two PIR sensors of the device 2. In this example, the number of detection areas 10 is higher than the number of PIR sensors used to monitor these detection areas 10. In particular, the number of PIR sensors in this example is s=a+b and the number of detection areas 10 associated with two or more of the PIR sensors is d=a×b, wherein a and bare natural numbers. In the example of FIG. 3, a=b=4. In other examples, a=1, 2, 3, 4, 5, 6, 7 or 8 and b=1, 2, 3, 4, 5, 6, 7 or 8 (e.g., a=b=8). In further examples, a #b (e.g., a=2 and b=3).

Of course, other shapes of the detection areas 10 and of the spatial areas 32, 36 compared with FIG. 3 are also possible. For example, a rectangular detection area may have a height to width ratio unequal to 1, may be rounded or hexagonal. The spatial axes 32, 38 may intersect at an angle different from 90° (e.g., an angle between 30° and 75°). The grid pattern may have a trapezoidal shape and each of the detection areas 10 may have a trapezoidal shape. Other groupings of detection areas into a group of detection areas are also possible (e.g., only the detection areas 10Ab, 10Bb and 10 Db, or the detection areas 10Ab, 10Ac, 10Bb and 10Aa may for a respective group).

A group of detection areas may consist of detection areas 10 that cover a continuous or coherent spatial portion. In this case, the detection areas 10 of that group may be adjacent to one another, border one another and/or be joined together, as shown in FIG. 3. Alternatively, a group of detection areas may consist of detection areas 10 that cover an interrupted spatial portion of a plurality of distinct, disjoint spatial portions. In this case, a first subgroup of the detection areas 10 of that group may be spatially offset from a second subgroup of the detection areas 10 of that group.

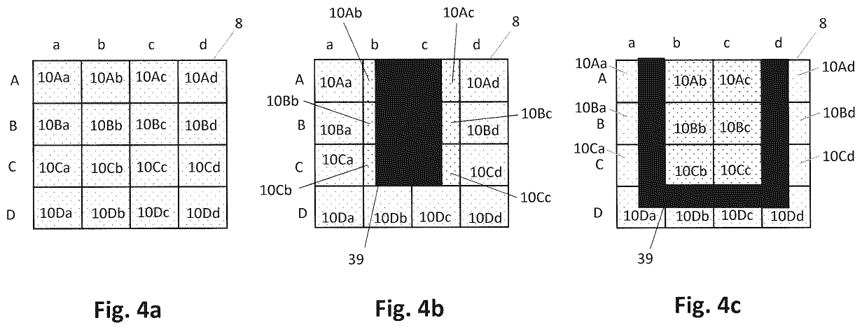

FIG. 4a-4c show three examples of detection areas 10. The example of FIG. 4a corresponds to the detection areas 10 as illustrated in FIG. 3. In this case, the detection areas 10 cover the complete patient zone 8. On the other hand, the examples of FIG. 4b-4c illustrate that the overall spatial detection range of the PIR sensors may comprise blind spots or blind regions 39. Put differently, certain parts of the patient zone 8 may be (e.g., intentionally) blocked out such that they are not observed by the PIR sensors of the device 2. In the example of FIG. 4b, a central portion of the patient zone 8 is blocked out as a rectangular blind region 39. The central portion may correspond to an area occupied by the patient bed 6. This may help to differentiate, based on detection signals of the PIR sensors of the device 2, a situation in which a patient has moved within the patient bed 6 or removed his blanked from a situation in which the patient has left the patient bed 6. In the example of FIG. 4c, a U-shaped portion of the patient zone 8 is blocked out as a blind spot 39. The U-shaped portion may correspond to a region bordering or bounding the patient bed 6 in the room 4. This may help to differentiate, based on detection signals of the PIR sensors of the device 2, a situation in which a patient has left the patient bed 6. from a situation in which the patient has entered the patient bed 6. Other variants are also possible. For instance, the blind spots may be configured to cover electrical medical equipment present in the patient zone 8, a heater arranged in the room 4 or a window of the room 4.

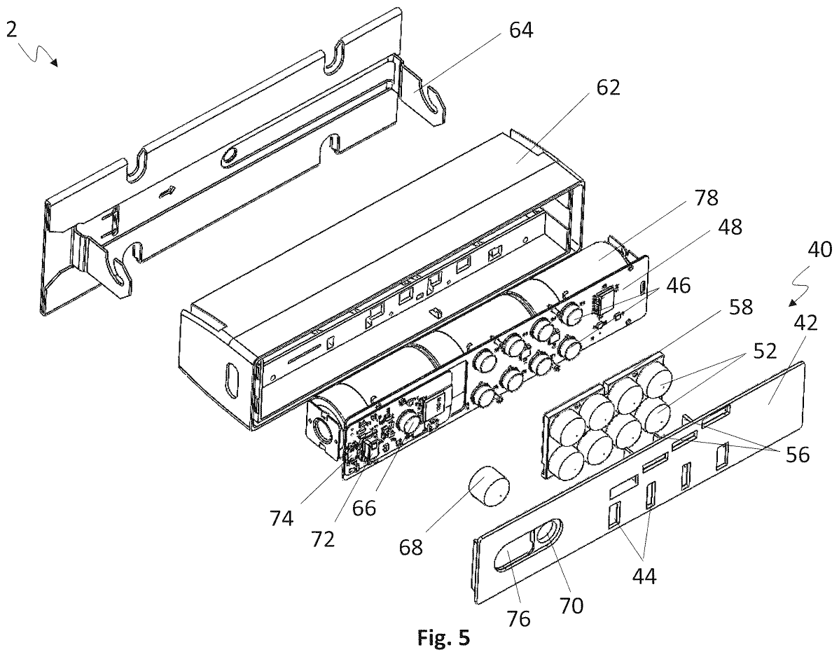

FIG. 5 shows an explosion drawing of the clinical monitoring device 2. The arrangement 40 in this example comprises a sheet 42 that is configured to block infrared light. The sheet 42 comprises a plurality of elongate slot-shaped through-holes, each configured as an optical aperture 44. Each of the apertures 44 is associated with a different one of the PIR sensors 46 arranged on a circuit board 48 of the clinical monitoring device 2. In particular, each of the apertures 44 defines an outer contour of the group (e.g., 28, 30) of detection areas associated with the respective PIR sensor 46. The slot-like shape of the apertures 44 in this example may lead to the rectangular contour of the spatial areas 32, 36 as shown in FIG. 3. Each of the PIR sensors 46 of the clinical monitoring device 2 may be associated with a different column or row of the detection areas 10 as shown in FIG. 3 and FIG. 4a. The PIR sensors 46 in the example of FIG. 5 are arranged in two rows of four sensors (4×2), whereas the groups of detection areas 10 may, as shown in FIG. 3, represent columns and rows of a grid arrangement of all (4×4) detection areas 10. Not only may the number of detection areas 10 observed by two or more PIR sensors 46 be higher than the total number of PIR sensors 46, the spatial arrangement (e.g., 4×2) of the PIR sensors 46 may also differ from the spatial arrangement (e.g., 4×4) of the groups (e.g., 28, 30) of detection areas 10. Other arrangements of the PIR sensors 46 are also possible, for example an arrangement of PIR sensors 46 in a single row or a n×m array of PIR sensors 46, wherein n and m are natural numbers and may or may not be equal to one another (e.g., n=2, 3, 4 or 5 and m=2, 3, 4 or 5). As an example, n=m=3.

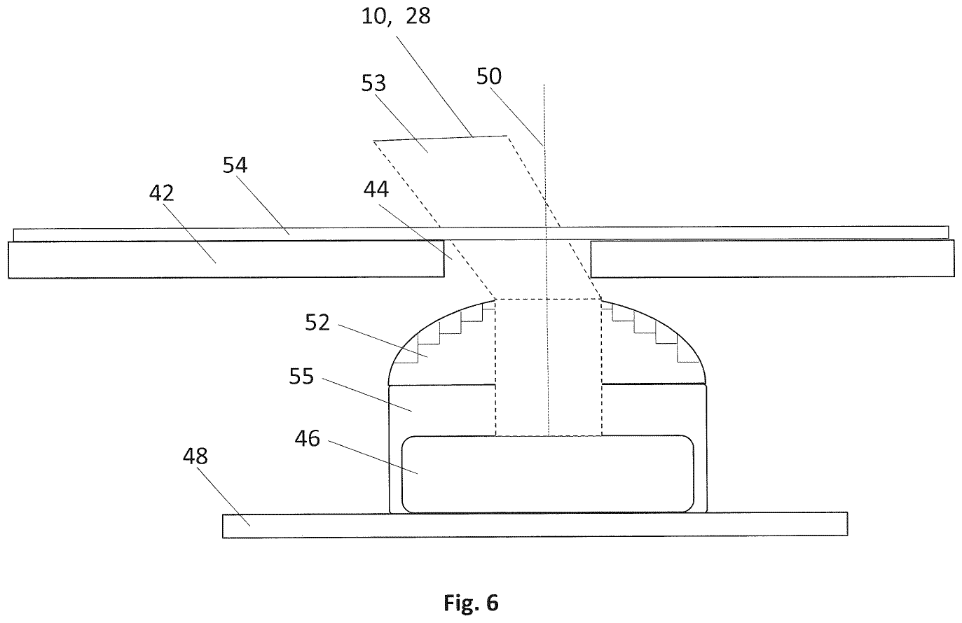

It can also be seen in FIG. 5 that the apertures 44 of the upper row are generally orthogonal to the apertures 44 of the lower row and that the apertures 44 are arranged with different spatial offsets relative to a central viewing axis of the respective PIR sensor 46. In the example of FIG. 5, adjacent apertures 44 are offset from one another by a distance that its larger than an offset between the associated adjacent PIR sensors 46 of a given column (e.g., the upper column of PIR sensors 46 or the lower column of PIR sensors 46 shown in FIG. 5). Due to these different orientations of the apertures 44 and the different offsets, the fields of view of the PIR sensors 36 differ from one another and the arrangement of detection areas 10 as illustrated in FIG. 3 and FIG. 4a may be obtained. This will now be explained with reference to FIG. 6.

FIG. 6 shows a schematic side view of a PIR sensor 46 of the clinical monitoring device 2. In this example, a center of the aperture 44 associated with the PIR sensor 46 is offset from the central detection or viewing axis 50 of the PIR sensor 46. The aperture 44, together with a (e.g., Fresnel) lens 52 arranged on the PIR sensor 46, defines the field of view, FoV, 53 of the PIR sensor 46 and, thus, the shape, position and orientation of (e.g., the group of) the detection areas 10 observed by the PIR sensor 46. The FoV 53 of the PIR sensor 46 is defined such that it covers the group of detection areas 10 associated with this PIR sensor 46, for example the group 28 covering the rectangular spatial area 32 as shown in FIG. 3. The further the aperture 44 is offset from the detection axis 50, the further will the detection areas 10 monitored by this PIR sensor 46 lie at the border of all detection areas 10 observed by the PIR sensors 46 of the device 2.

The lens 52 comprises a receptacle or blind hole 55 within which the PIR sensor 46 is located. One may say that the lens 52 houses the PIR sensor 46. The lens 52 may contact the circuit board 48. This may provide a predefined relative alignment between the lens 52 and the PIR sensor 46.

Also illustrated in FIG. 6 is an optional protective cover 54 configured to protect the PIR sensor 46 from an outer environment. The cover 54 extends through the FoV 53 and covers the aperture 44. The cover 54 is optically transparent (e.g., with a transmissibility of >70%), but only for infrared light (e.g., between 8 μm and 10.5 μm) and may not be transparent for light in the range of wavelengths visible to the human eye. Thus, a human may be able not see the PIR sensor 46 through the cover 54. The cover 54 may be a HDPE foil adhesively attached onto the sheet 42.

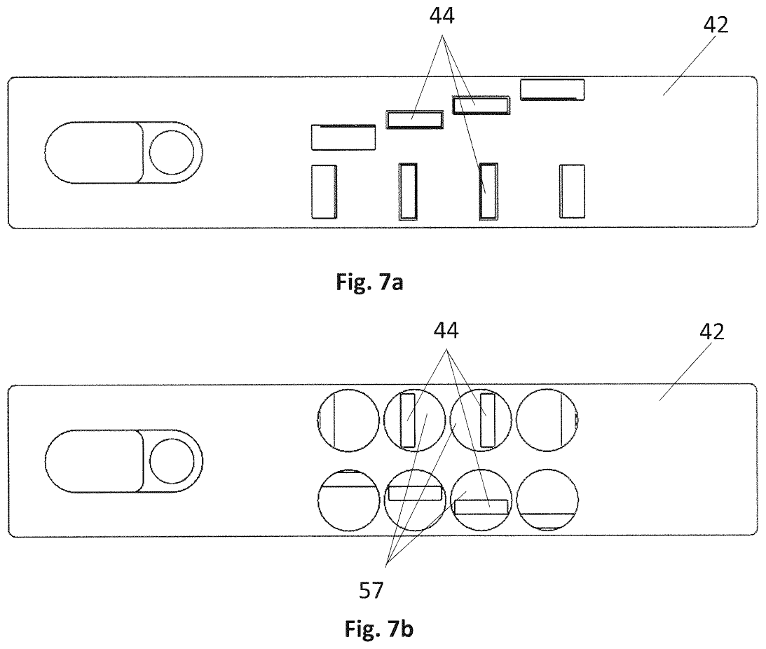

The sheet 42 may be configured as a replaceable or removable component of the device 2. FIG. 7a-7b show different sheets 42 that may be comprised in or used for the device 2. A clinical monitoring kit may be provided, which comprises the clinical monitoring device 2 and a plurality of the sheets 42, differing from one in at least one of (i) a number of apertures 44 formed in the respective sheet 42, (ii) a shape (e.g., contour or outline) of one or more apertures 44 formed in the respective sheet 42, (iii) a size of one or more apertures 44 formed in the respective sheet 42, (iv) a position and/or orientation of one or more apertures 44 formed in the respective sheet 42. The sheets of the kit may alternatively or additionally differ from one another in the (e.g., degree or type of) adjustability of the apertures 44.

In FIG. 7a, the sheet 42 is configured with predefined and fixed apertures 44, which are formed as through-holes, having rectangular outlines, through the sheet 42. Alternatively, the apertures 44 may be configured to be adjustable in shape, size, position and/or orientation.

In FIG. 7b, the apertures 44 are formed in a first sheet 57 that is separate from the sheet 42. The first sheet 57 may be a replaceable component and may be arranged in a fixed spatial relationship relative to the sheet 42 comprising (e.g., circular) through-holes. An overlap between the apertures 44 in the first sheet 57 and the circular through-holes in the sheet 42 may define the FoV 53 and, thus, the detection areas 10 of the respective PIR sensor 46.

The first sheet 57 may be movable relative to the sheet 42. By moving the first sheet 57 relative to the sheet 42, the offset of the apertures 44 relative to the central detection axes 50 of the PIR sensors 46 will change accordingly, thereby shifting the detection areas 10 relative to the room 4. An actuator (not shown) may be provided in the device 2 to adjust the apertures 44, for example by moving the first sheet 55 relative to the sheet 42.

It is to be understood that instead of providing a first sheet 57 and/or the sheet 42 with permanently shaped apertures 44, shape-configurable apertures 44 (e.g., in the form of collimators or as known from photography apparatuses) may be used as adjustable apertures 44. In this case, the actuator may be configured to adjust a shape (e.g., contour or outline), size, position and/or orientation of one or more of the apertures 44 (e.g., relative to the associated PIR sensor 46).

Although the apertures 44 illustrated in FIGS. 4, 7a and 7b are elongate rectangular slots, other geometrical forms are also possible. For example, an aperture 44 may be circular, elliptical, trapezoidal, square, hexagonal or have a rounded shape (e.g., a U-shape). The size, shape, position and orientation of the respective apertures 44 may differ from one another and/or may be selected (e.g., predefined or adjusted) based on the detection areas 10 to be monitored by the PIR sensors 10 and based on the spatial arrangement of the PIR sensors 10 on the circuit board 48.

It is also possible to provide an aperture 44 that is commonly used by two or more PIR sensors 46. Alternatively, or in addition, a PIR sensor 46 may be associated with two or more of the apertures 44. This may enable the provision of blind spots or blocked areas as described above with reference to FIGS. 4b and 4c. Referring to FIG. 4b, a first aperture 44 of the PIR sensor 46 associated with detection areas 10Aa, 10Ab, 10Ac and 10Ad may define the narrow rectangular detection area 10Aa, a second aperture 44 associated with the same PIR sensor 46 may define the square detection areas 10Ab and 10Ac, and a third aperture 44 associated with the same PIR sensor 46 may define the narrow rectangular detection area 10Ad.

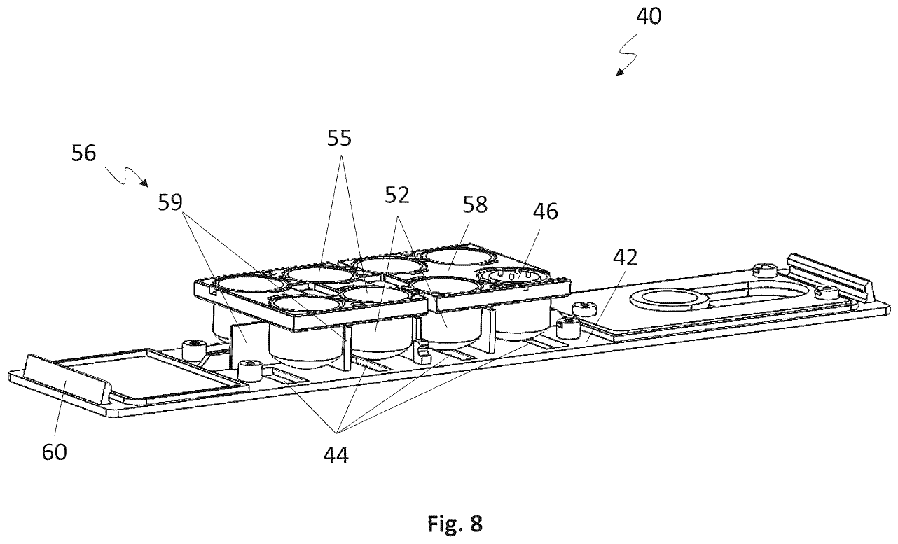

As illustrated in FIG. 8, the arrangement 40 further comprises an optional light-blocking assembly 56 configured to block (e.g., stray-)light falling in through one of the apertures 44 from reaching one or more of the PIR sensors 46 that are not associated with the one of the apertures 44. The light-blocking assembly 56 in this example comprises several walls 59 opaque for infrared light, which are arranged between adjacent apertures 44 on the sheet 42 or 57.

The arrangement 40 shown in FIG. 8 further comprises a set of lenses 52, each being associated with a different one of the PIR sensors 46. The lenses 52 may be attached to a lens carrier 58 to ensure that they maintain a predefined relative alignment. The lenses 58, together with the apertures 44, define the respective detection areas 10 and groups of detection areas (e.g., 28, 32) for the PIR sensors 46.

The lens carrier 58 may be configured to arrange the lenses 52 in a predefined pose relative to the apertures 44. For example, the lens carrier 58 may be mechanically coupled to the sheet 42 and/or the walls of the light blocking assembly 56. In FIG. 8, one of the PIR sensors 46 is illustrated without the circuit board 48. The lenses 52 carried by the lens carrier 58 are arranged such that each of the PIR sensors 46 is located within the room 55 of the associated lens 52 in the assembled state of the device 2. The sheet 42 may comprise a mechanical (e.g., snap-fit) coupling 60 to mount the sheet 42 onto a housing 62 of the device 2 (see FIG. 4) in a predefined pose. The circuit board 48 and the PIR sensors 46 may be arranged within this housing 62, as indicated in FIG. 5.

It is also possible that the arrangement 40 comprises a lens that is associated with two or more of the PIR sensors 46. The arrangement 40 may comprise either the lenses 52 or the apertures 44 or both. In other words, the arrangement 40 may consist of one or more lenses (i.e., no apertures 44), may consist of one or more apertures (i.e., no lens(es) 52), or may comprise the lenses 52 and the apertures 44.

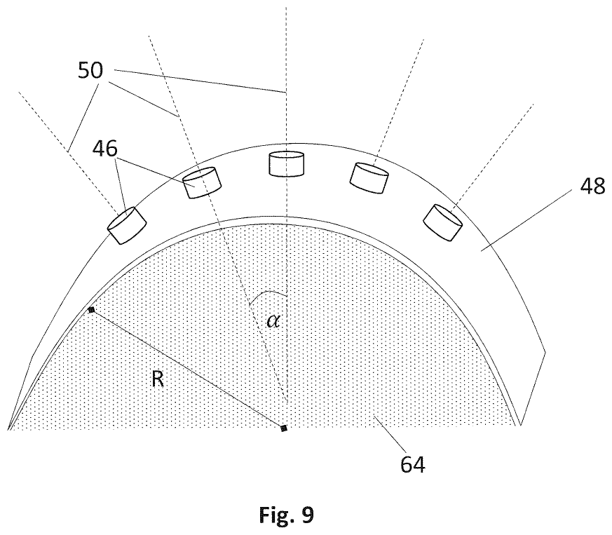

In one variant illustrated in FIG. 9, the circuit board 48 is a flexible substrate carrying the PIR sensors 46. The arrangement 40 in this case may comprise a shape-defining member 64 (e.g., a metal block or a polymeric wedge) that deforms the flexible substrate into a predefined shape having a predefined bending radius R, such that the central detection axes 50 of adjacent PIR sensors 46 differ from one another by a predefined angular offset α. This arrangement may obviate the need for an aperture 44 or a lens 52, as the detection areas 10 may then be defined solely by the shape-defining member 64. Of course, a combination of the shape-defining member 46 and the flexible substrate with apertures 44 and/or lenses 52 is also possible. A clinical monitoring bundle may be provided, comprising the clinical monitoring device 2 and a plurality of the shape-defining members 46, differing from one another in the predefined shapes into which they deform the flexible substrate.

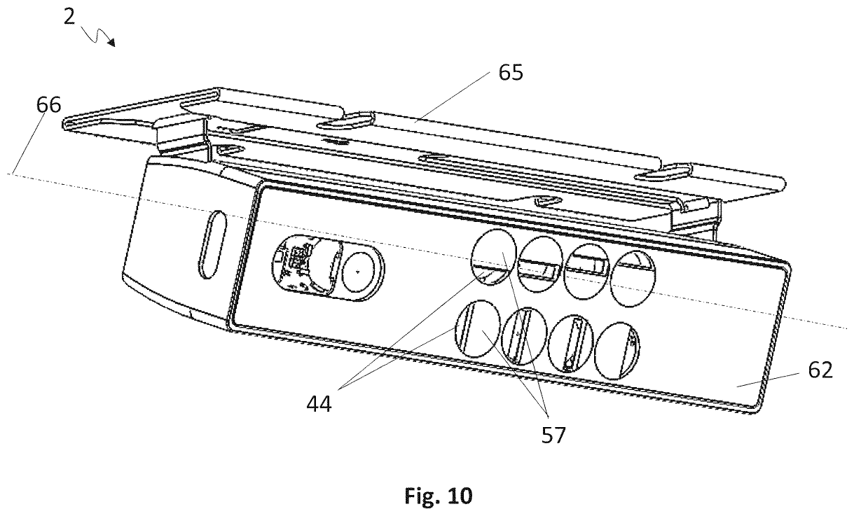

Referring back to FIG. 1, to shift the detection areas 10 relative to the hospital room 4, for example in case a patient bed 6 was moved outside the patient zone 8 covered by the detection areas 10, the device 2 may be tilted relative to the hospital room 4. To this end, the device 2, as shown in FIG. 5 and FIG. 10, comprises a mounting arrangement 65 configured to rotatably mount the housing 62 of the device 2 to a ceiling or wall of the hospital room 4. As shown in FIG. 10, the mounting arrangement 65 may enable the housing 62 of the device 2 to be rotated (e.g., only) around a rotation axis 66 relative to a ceiling or wall of the hospital room 4 to which the mounting arrangement 65 is attached.

The clinical monitoring device 2 may comprise additional sensors. In the example of FIG. 5, the clinical monitoring device 2 comprises an additional PIR sensor 66, having a FoV covering a spatial portion adjacent to at least some of the outermost detection areas 10 of the plurality of PIR sensors 46. That is, the additional PIR sensor 66 may have a FoV that covers not only the patient zone 8, but also a region around the patient zone 8. This may enable detecting a temperature change outside the patient region 8 with the additional PIR sensor 66, even before detecting a temperature change with the PIR sensors 46 (e.g., in case a person walks toward the patient zone 8). The additional PIR sensor 66 may be associated with a lens 68 and an aperture 70 formed in the sheet 42. The aperture 70 may be larger than the apertures 44 to provide a larger FoV for the PIR sensor 66 compared with the PIR sensors 46. The optional cover 54 may extend across the aperture 70. The PIR sensor 68 may be referred to as wake-up sensor and may be used to activate certain components of the device 2 from a standby state to an active state upon detecting a temperature change exceeding a predefined detection threshold.

The clinical monitoring device 2 may further comprises a thermal sensor arrangement 72 (e.g., a thermal camera) configured to provide spatially resolved temperature measurements of a spatial sector comprising at least one of the detection areas 10, and, optionally, a distance sensor arrangement 74 (e.g., a time-of-flight, ToF, sensor or a stereo camera) configured to provide spatially resolved distance measurements of a spatial field comprising at least one of the detection areas 10. The spatial sector may correspond to the spatial field, and both may overlap, correspond to or comprise the patient zone 8. Each of the arrangements 72, 74 may be associated with a respective aperture or a common aperture 76 provided in the sheet 42. The optional cover 54 may extend across (e.g., cover) the respective aperture of common aperture 76.

As shown in FIG. 5, the device 2 may further comprise an energy supply 78 (e.g., in the form of replaceable batteries). It is also possible to provide the device 2 with a cable-bound energy supply, although this may increase installation costs. The housing 62 may be removable from the mounting arrangement 65 for maintenance purposes (e.g., inspection or exchange of the batteries 78).

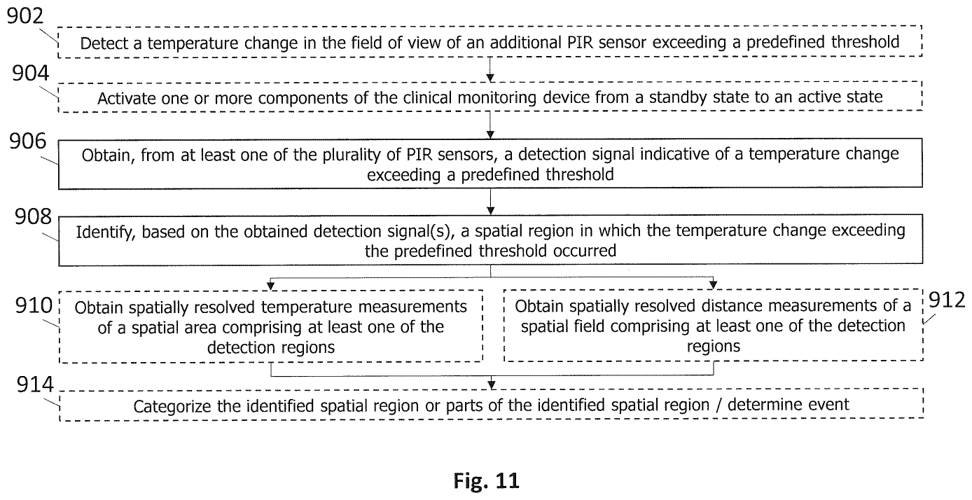

FIG. 11 illustrates a method in accordance with the present disclosure. The method may be performed by the processor 24 or by the processor 16.

In an optional step 902, it is detected, with the additional PIR sensor 66, that a temperature change in the FoV of the additional PIR sensor 66 occurred that exceeded a predefined (e.g., first) threshold. The first threshold may correspond to the minimal temperature change that is detectable by the additional PIR sensor 66. The first threshold may be adjustable and/or selected such that unwanted detections (e.g., due to thermal noise) are minimized or avoided.

Responsive to the detecting, in optional step 904, one or more components of the clinical monitoring device 2 (e.g., the sensor arrangements 70, 72 and the PIR sensors 46) are activated from a standby state to an active state.

In step 906, from at least one of the PIR sensors 46, a detection signal is obtained which is indicative of a temperature change within one of the detection areas 10 associated with the at least one PIR sensor 46 exceeding a predefined (e.g., second) threshold. The second threshold may correspond to the minimal temperature change that is detectable by the at least one PIR sensor 46. The second threshold may be adjustable and/or selected such that unwanted detections (e.g., due to thermal noise) are minimized or avoided. The second threshold may correspond to the first threshold.

In step 908, based on the obtained detection signal(s) (e.g., further based on a known association between the respective PIR sensors 46 and the detection regions 10), a spatial region is identified in which the temperature change exceeding the predefined (e.g., second) threshold occurred.

The detection signals may be obtained from the two or more of the PIR sensors 46 that are associated with a similar detection area (e.g., 10Bb). In this case, the similar detection area (e.g., 10Bb) may be identified as (e.g., at least a part of) the spatial region (e.g., based on the known association between the respective PIR sensors 46 and the detection regions 10). As another example, one PIR sensor 46 may be associated with exactly one detection area (e.g., 10Ab) and may detect a temperature change exceeding the predefined (e.g., second) threshold. In this case, the detection area 10 associated with this particular PIR sensor 46 may be identified as (e.g., at least a part of) the spatial region. In case a detection area 10 is monitored by multiple PIR sensors 46 but only one of these PIR sensors 46 detects a temperature change exceeding the predefined (e.g., second) threshold, this detection may be neglected and the detection area 10 monitored by the multiple PIR sensors 46 may not be identified as part of the spatial region. In case a temperature change exceeding the predefined (e.g., second) threshold is detected by multiple PIR sensors 46 but with a temporal offset from one another that exceeds a predefined first time window, the detection area (e.g., 10Bb) associated with these multiple PIR sensors 46 may also not be identified as part of the spatial region. Detections of temperature changes exceeding the predefined (e.g., second) threshold within a given detection area 10, which detections occur within the predefined first time window, may be considered as “simultaneous detections” by multiple sensors 46. In case a detection area 10 is monitored by multiple PIR sensors 46, this detection area 10 may only be identified as (e.g., at least a part of) the spatial region if “simultaneous detections” occurred for this detection areas 10.

The spatial region may consist of all detection areas in which simultaneous detections were detected within a predefined second time window. In other words, the first time window may be used for detecting “simultaneous detections” within a particular detection area, whereas the second predefined time window may be used for selecting all the detection areas in which the simultaneous detections were observed, which simultaneous detections fall into the same second time window.

One may say that in a first (e.g., preprocessing) step, the detection signals are used to determine one or more simultaneous detections using the first time window, whereas, in a second step, the determined simultaneous detections are used to identify the spatial region by selecting all detection areas associated with simultaneous detections as part of the spatial region. This use of the first and the second time windows may be referred to as double moving windows-processing, as both windows may be moved across the detection signal(s) over time (e.g., for detecting peaks in the detection signals and classifying peaks falling into the first time window as representing a simultaneous detection if the respective PIR sensors 46 are associated with a same detection area 10).

The first predefined time window may be (e.g., two to fifteen times, for example three to ten times, or four to ten times) smaller (e.g., shorter) than the second predefined time window. Only as an example, the first predefined time window may have a length between 500-2000 ms and the second predefined time window may have a length between 2 s-5 s. Other lengths of the time windows may be chosen (e.g., depending on the type of PIR sensors 46 and the processing capabilities of the at least one processor performing the identification of the spatial region).

In optional step 910, the spatially resolved temperature measurements are obtained from the thermal sensor arrangement 72.

In optional step 912, the spatially resolved distance measurements are obtained from the distance sensor arrangement 74.

The temperature measurements and/or the distance measurements may be spatially mapped to the detection signal(s) obtained in step 906 or to the identified spatial region. The field of view of the thermal sensor arrangement 72 and the field of view of the distance sensor arrangement 74 may be mapped to the detection areas 10. One may say that the detection areas 10 or the identified spatial region may be enriched with measurements from the thermal sensor arrangement 72 and/or the distance sensor arrangement 74.

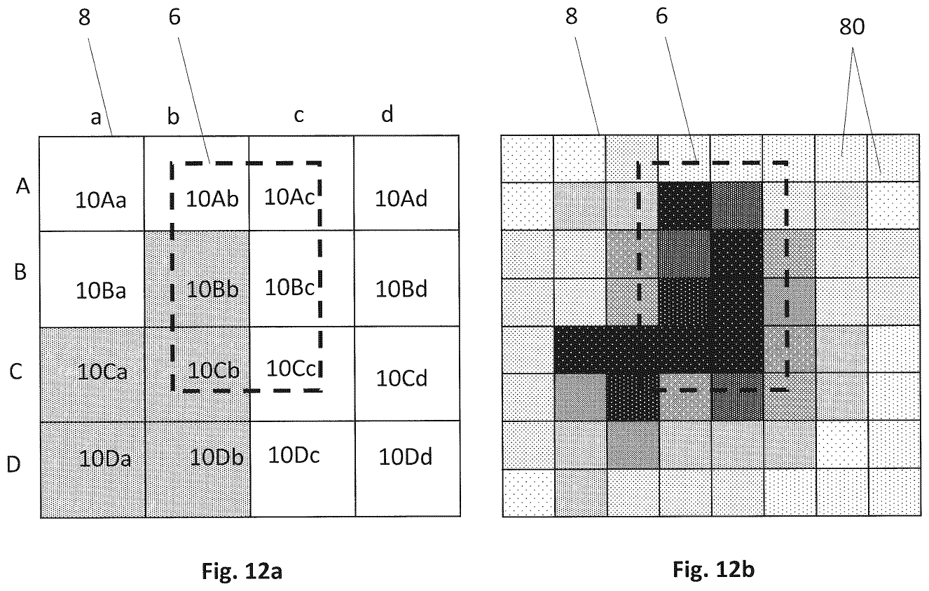

In optional step 914, the identified spatial region or parts thereof are categorized or classified (e.g., into a set of predefined categories or classes) based on the temperature measurements and/or the distance measurements. The classification may be performed by a trained machine learning model. Instead of, or in addition to classifying the identified spatial region or parts thereof, an event may be determined based on the identified spatial region and, optionally, based on the temperature measurements and/or the distance measurements. The event may be selected from a list of predetermined events. The event may be indicative of one or more predefined activities (e.g., a patient entering the patient bed 6, a person leaving the patient zone 8 or the like). The determination of the event may be performed by a trained machine learning model. Based on the determined event, a recommended action may be determined (e.g., a recommendation for a person, having entered the patient zone 8 until coming into close proximity of the (e.g., infectious) patient in the patient bed 6, to disinfect his/her hands to comply with hygiene rules). The optional steps 910-914 will now be explained with reference to FIG. 12a-12b.

FIG. 12a visualizes the detection areas 10 of FIG. 3 and FIG. 4a together with an indication of the patient bed 6. Detection areas 10 in which a temperature change exceeds the predefined threshold are highlighted. That is, in this example, the obtained detection signals indicate that a temperature in the detection areas 10Bb, 10Ca, 10Cb, 10Da and 10 Db exceeded the predefined (e.g., second) threshold. Each PIR sensor 46 may have a cooldown time or a minimum detection interval of around 200 ms-500 ms. For example, the PIR sensor 46 associated with the detection areas of row D and the PIR sensor 46 associated with the detection areas of column a may each detect a temperature change exceeding the predefined (e.g., second) threshold detection event within the predefined first time window, as a simultaneous detection. Based on this simultaneous detection, it can be derived that a temperature change occurred within the detection area 10Da, which exceeded the predefined (e.g., second) temperature threshold. The same applies for the detection areas 10Ca, 10Cb, 10Db and 10Bb, respectively.

Simultaneous detections may also be observed in other detection areas (e.g., 10Ba and 10Aa). In the illustrated example, these other detection areas are not identified as part of the spatial region, as their respective simultaneous detections occurred at a time that falls outside the second time window, whereas the respective simultaneous detections in the detection areas 10Bb, 10Ca, 10Cb, 10Da and 10Db occurred within the second time window and the detection areas 10Bb, 10Ca, 10Cb, 10Da and 10Db are thus identified as part of the spatial region. One may say that the highlighted detection areas of FIG. 12a represent the areas in which simultaneous detections were observed within the last (e.g., the previous) time period, wherein the length of the last time period corresponds to the second time window.

To recap, based on the detection signals, one or more (e.g., all) of the detection areas 10Bb, 10Cb, 10Ca, 10Da and 10Db may be identified as the spatial region. Although this information helps in locating a temperature change within the room 4, further information may be used to classify the identified spatial region or to determine presence of an event. For example, if only the information provided by the PIR sensors 46 was used, it may be ambiguous whether a person has exited the patient zone 8 or whether a person has entered the patient zone 8, which activities may both result in the same identified spatial region shown in FIG. 12a. Sensor data representing a current (and, optionally, a previous) state of the patient zone 8 may be used to enable a more precise categorization and/or determination of the event. For example, sensor data from the arrangement 72 representative of a current (and, optionally, a previous) spatial temperature distribution within the patient zone 8 may be used to this end, or sensor data from the arrangement 74 representative of a current (and, optionally, a previous) surface topology of the patient zone 8.

FIG. 12b visualizes detection areas 80 of the thermal sensor arrangement 72. Detection areas 80 in which a higher temperature was detected by the thermal sensor arrangement 72 are shaded darker than detection areas 80 in which a lower temperature was detected by the thermal sensor arrangement 72. It can be seen that in comparison with the overall patient zone 8, relatively high temperatures were detected in detection areas 80 that overlap with the patient bed 6. This leads to the conclusion that a patient is present within the patient bed 6. Some detection areas 80 adjacent to the patient bed 6 also exhibit relatively high temperatures. This means that a heat dissipating object is present next to the patient bed 6.

By spatially mapping the information provided by the PIR sensors 46 as shown in FIG. 12a to the temperature information shown in FIG. 12b, it is possible to classify at least the detection areas 10Ca, 10DAa, 10Bb, 10Cb and 10Db of the PIR sensors 46. In particular, the combination of both types of information indicates that a person entered the room 4 from the left bottom side (detection area 10Da) and is now located next to the patient bed 6. By combining the information obtained from the PIR sensors 46 with the information obtained from the sensor arrangement 72 and/or 74, it may be determined (e.g., as the activity represented by the respective event, or as the event) whether a person entered the patient zone 8, whether a person left the patient zone 8, whether the patient entered the patient bed 6, whether the patient left the patient bed 6, whether the patient lying in the bed 6 covered himself with a blanket, whether a medical device located next to the patient bed 6 shows an unwanted heating behavior, or the like.

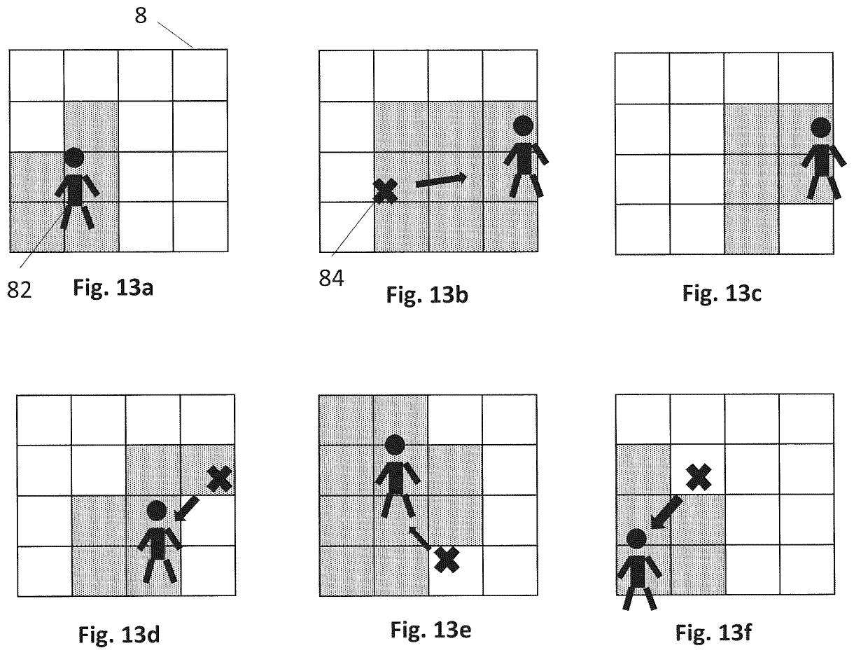

At least steps 906 and 908 may be performed multiple times (e.g., iteratively at different points in time). This may lead to a plurality of identified spatial regions which differ from one another over time. Not only may additional simultaneous detections be observed over time, but the second time window, when shifted in time (e.g., to cover only the previous 2 -5 s), may lead to different combinations of detection areas 10 in which simultaneous detections occurred (due to temporal offsets between the simultaneous detections) and, thus, to different identified spatial regions. The change of the identified spatial region over time may be used to determine a movement of a heat dissipating object such as a person in the room 4. This is illustrated in FIG. 13a-13f, which show a temporal sequence of a person 82 moving through the patient zone 8, and the resulting change of the identified spatial region. In FIG. 13a, the person 82 has entered the patient zone 8. The person 82 then moves through the room 4 to an opposite side thereof, as shown in FIG. 13b-13c. As shown in FIGS. 13d-13f, the person 82 then moves throughout the room 4 and finally leaves the patient zone 8. It can be seen that the person 82 triggers detections at multiple detection areas 10. To determine a movement direction of the person in the room 4, a center 84 of the identified spatial region(s) may be tracked over time.

The technique disclosed herein may enable monitoring of a patient zone with a low-cost clinical monitoring device 2, using low computing resources and a low power consumption by the device 2. The PIR sensors 46, as passive sensors, generally require little energy for the detection of temperature changes in the detection areas 10 (e.g., a total of ~40 μA, which is much less than the ~40-100 mA required by typical forward looking infrared, FLIR, modules and even less than the ~4500 μA required by a typical low cost, low pixel thermal camera). The arrangement 40 defines the groups of detection areas 10 associated with the individual PIR sensors 46 such that a relatively low number of PIR sensors 46 is required while still enabling a relatively high spatial resolution for the identified spatial region. The number of PIR sensors 46 may be much lower than the number of detection areas 10 associated with different pairs of the PIR sensors 46. By providing lenses 52, apertures 44 and/or a shape-defining member 64, a reliable definition of the detection areas 10 may be ensured at low manufacturing cost while optimizing the detection capabilities of the device 2. Additional sensors of the device 2 may be used to enrich the detection data from the PIR sensors 46, thereby enabling a classification of the detection areas 10 or a determination of a presence of an event (e.g., a person having entered the patient zone 8 and now standing at the side of the patient bed 6). The mounting arrangement 65, adjustable lenses 52, adjustable apertures 44, a set of different shape-defining members 64 and/or a set of different sheets 42 or 57 may allow for an easy change of the detection areas 10 in size, shape, position and/or orientation (e.g., relative to the hospital room 4).

In addition to the variants and options described herein, further modifications of the technique are possible. For instance, the clinical monitoring device 2 may be mounted on a pole attached to or standing on the floor of the room 4. As another example, it is also possible to use data obtained from the device 2 (e.g., based on the detection signal(s) of the at least one PIR sensor 46) in conjunction with data obtained from medical surveillance equipment (e.g., a heart rate monitoring system, a body temperature monitoring system, a breathing rate monitoring system or the like) to determine the presence of an event or to determine a recommendation (e.g., a medical alert or the need for clinical staff to check on the patient in the patient bed 6). Further advantages and possible modifications of the technique described herein will become apparent to those skilled in the art.

1. A clinical monitoring device, comprising:
a plurality of passive infrared, PIR, sensors for monitoring a patient zone; and
an arrangement configured to define a plurality of detection areas for the PIR sensors, wherein at least one of the detection areas is associated with two or more of the PIR sensors,
wherein the detection areas associated with a respective one of the PIR sensors form a group of detection areas covering an elongate spatial area extending longitudinally along a spatial axis,
wherein the spatial axis; differs between two or more of the groups of detection areas.

2-4. (canceled)

5. The clinical monitoring device of claim 1, wherein the detection areas are arranged in a grid pattern.

6-11. (canceled)

12. The clinical monitoring device of claim 1, wherein the number d of the at least one detection area associated with two or more of the PIR sensors is d=a×b, wherein the number s of PIR sensors is s=a+b.

13. The clinical monitoring device of claim 1, wherein the arrangement comprises one or more optical apertures.

14. The clinical monitoring device of claim 13, wherein each of the apertures is associated with a different one of the PIR sensors and defines at least an outer contour of (i) the detection areas associated with the one PIR sensor or (ii) the group of detection areas associated with the one PIR sensor.

15-17. (canceled)

18. The clinical monitoring device of claim 13, wherein one or more of the apertures has a spatial offset from a central detection axis of the PIR sensor associated with the respective aperture.

19-20. (canceled)

21. The clinical monitoring device of claim 13, wherein one or more of the apertures is adjustable in at least one of size and/or position relative to the associated PIR sensor.

22. The clinical monitoring device of claim 21, wherein the arrangement comprises at least one actuator configured to adjust the one or more of the apertures in at least one of size and/or position relative to the associated PIR sensor.

23. The clinical monitoring device of claim 13, wherein the arrangement comprises a sheet in which one or more of the apertures is formed as a through-hole.

24. (canceled)

25. The clinical monitoring device of claim 13, wherein the arrangement further comprises a light-blocking assembly configured to block light falling in through one of the apertures from reaching one or more of the PIR sensors that are not associated with the one of the apertures.

26. The clinical monitoring device of claim 25, wherein the light-blocking assembly comprises one or more walls configured to extend between adjacent PIR sensors.

27-28. (canceled)

29. The clinical monitoring device of claim 1, further comprising:
a protective cover configured to protect the PIR sensors from an outer environment.

30. The clinical monitoring device of claim 29, wherein the protective cover is arranged within a field of view of one or more of the PIR sensors.

31-34. (canceled)

35. The clinical monitoring device of claim 1, wherein the arrangement comprises two or more lenses, each being associated with a different one of the PIR sensors.

36-38. (canceled)

39. The clinical monitoring device of claim 1, further comprising at least one of the following components:
a mounting arrangement configured to tiltably mount the device to a ceiling or wall of a hospital room such that the detection areas can be shifted relative to the hospital room by tilting the device;
an additional passive infrared, PIR, sensor having a field of view covering a spatial portion adjacent to at least some of the outermost detection areas of the plurality of PIR sensors;
a thermal sensor arrangement configured to provide spatially resolved temperature measurements of a spatial sector comprising at least one of the detection areas;
a distance sensor arrangement configured to provide spatially resolved distance measurements of a spatial field comprising at least one of the detection areas;
a wired or wireless interface configured to output data based on signals of the plurality of PIR sensors.

40-47. (canceled)

48. A clinical monitoring method using a clinical monitoring device comprising (i) a plurality of passive infrared, PIR, sensors for monitoring a patient zone, and (ii) an arrangement configured to define a plurality of detection areas for the PIR sensors, wherein at least one of the detection areas is associated with two or more of the PIR sensors wherein the detection areas associated with a respective one of the PIR sensors form a group of detection areas covering an elongate spatial area extending longitudinally along a spatial axis, wherein the spatial axis differs between two or more of the groups of detection areas, the method comprising:
obtaining, from at least one of the PIR sensors, a detection signal indicative of a temperature change exceeding a predefined threshold; and
identifying, based on the obtained detection signal(s), a spatial region in which the temperature change exceeding the predefined threshold occurred.

49. The method of claim 48, wherein
the detection signals are obtained from the two or more of the PIR sensors associated with the at least one of the detection areas and are indicative of a temperature change within the at least one of the detection areas exceeding the predefined threshold, wherein the at least one of the detection areas is identified as at least a part of the spatial region,
wherein the temperature change indicated by the obtained detection signals is observed by the two or more of the PIR sensors within a predefined time window.

50. (canceled)

51. The method of claim 48, wherein the spatial region is identified for different points in time, wherein the method comprises:
determining a movement of a heat-dissipating object or person based on a change of the identified spatial region over time.

52. The method of claim 48, the method further comprising one of:
(i) obtaining spatially resolved temperature measurements of a spatial sector comprising at least one of the detection areas, and categorizing the identified spatial region or parts of the identified spatial region based on the spatially resolved temperature measurements; or
(ii) obtaining spatially resolved distance measurements of a spatial field comprising at least one of the detection areas, and categorizing the identified spatial region or parts of the identified spatial region based on the spatially resolved distance measurements.

53. (canceled)

54. The method of claim 48, further comprising:
detecting, with an additional passive infrared, PIR, sensor having a field of view covering a spatial portion adjacent to at least some of the outermost detection areas of the plurality of PIR sensors, a temperature change in the field of view exceeding a predefined threshold; and
responsive to the detecting, activating one or more components of the clinical monitoring device from a standby state to an active state.

55-60. (canceled)
